(19) United States
(12) Patent Application Publication
(10) Pub. No.: US 2017/0369836 A1
(43) Pub. Date: Dec. 28, 2017

(54) SERUM-FREE MEDIUM FOR FULL SUSPENSION CULTURE OF MDCK CELLS AND PREPARATION METHOD OF SERUM-FREE MEDIUM

(71) Applicants: Zhaoqing Dahuanong Biological Medicine Co., Ltd, ZHAOQING (CN); East China University of Science and Technology, SHANGHAI (CN); Guangdong Wens Dahuanong Biotechnology Co., Ltd., Yunfu (CN)

(72) Inventors: RUIAI CHEN, ZHAOQING (CN); Hanzhang Lai, ZHAOQING (CN); Wensong Tan, SHANGHAI (CN); Xuanzi Zhan, ZHAOQING (CN); Xuping Liu, SHANGHAI (CN); Kangcong MAI, ZHAOQING (CN); Weilan Pan, ZHAOQING (CN); Xiaofen Wang, ZHAOQING (CN); Huajian Chen, ZHAOQING (CN); Peijun Chen, ZHAOQING (CN); Jiahua Xu, ZHAOQING (CN); Qin Tang, YUNFU (CN); Wenyan Zhang, ZHAOQING (CN); Yan Gao, ZHAOQING (CN)

(73) Assignees: Zhaoqing Dahuanong Biological Medicine Co., Ltd; East China University of Science and Technology; Guangdong Wens Dahuanong Biotechnology Co., Ltd.

(21) Appl. No.: 15/619,515

(22) Filed: Jun. 11, 2017

(30) Foreign Application Priority Data
Jun. 24, 2016 (CN) 201610486303.8

(51) Int. Cl. C12N 5/00

(52) U.S. Cl. CPC C12N 5/00; C12N 2500/90; C12N 2500/05; C12N 2500/40; C12N 2500/38; C12N 2500/36; C12N 2500/34; C12N 2500/32; C12N 2500/30; C12N 2500/24; C12N 2500/22; C12N 2500/20; C12N 2500/16; C12N 2500/14; C12N 2500/12; C12N 2500/76

(57) ABSTRACT

The present invention discloses a serum-free medium for full suspension culture of MDCK cells and a preparation method of the serum-free medium. The serum-free medium for full suspension culture of the MDCK cells comprises basic metabolic nutrients, nucleotide, vitamins, inorganic salts, a shear force protective agent, a cell clustering resisting agent, a pH buffer agent, a pH indicator, an influenza virus proliferation accelerant and other additives. The preparation method of the serum-free medium for the full suspension culture of the MDCK cells comprises the following steps: 1) preparing a mixed solution: dissolving and mixing raw materials; and 2) regulating pH: regulating the pH of the mixed solution to 6.3 to 6.7, and setting a constant volume to obtain the serum-free medium for the full suspension culture of the MDCK cells. The medium supports the high-density full-suspension culture of the MDCK single cells, greatly shortens a time for educating the MDCK cells from adherent cells into the serum-free full suspension cells, and is applicable to the mass production of biological products, and particularly veterinary biological products.

### CROSS-REFERENCE TO RELATED APPLICATIONS

This application claims priority to Chinese Patent Application No. 201610486303.8 with a filing date of Jun. 24, 2016. The content of the aforementioned application, including any intervening amendments thereto, are incorporated herein by reference.

### TECHNICAL FIELD

The present invention relates to the field of the preparation of a serum-free medium, and in particular relates to a serum-free medium for full suspension culture of MDCK cells and a preparation method of the serum-free medium.

### BACKGROUND OF THE PRESENT INVENTION

Madin-Darby canine kidney (MDCK) cells are regarded as one of cell lines most suitable for producing influenza A and B virus vaccines and may be used to substitute chick embryo to culture the influenza virus. At present, a micro-carrier adherent culture production method utilizing an MDCK cell line to substitute the chick embryo to culture the influenza virus has already been developed. Although the technological method can reach certain production scale, the method still has some defects; 1. micro-carriers are difficult to reuse repeatedly, resulting in high production cost; 2. the culture density of adherent cells is limited by an adherent area, resulting in low yield of the viruses; and 3. the adherent culture generally needs to add the serum to help the attachment and growth of the cells and needs the medium change, so the process is complicated, and mycoplasma, chlamydia or animal protein may be introduced to cause the pollution, thereby posing a potential threat to the safety of vaccine products. People pay more attention to the large-scale serum-free full suspension culture technology of the MDCK cells. Reports about the full suspension culture of the MDCK cells in a serum-free medium are rare. At present, commercial serum-free media suitable for the large-scale full suspension culture of the MDCK cells are extremely rare in China. In the prior art, the serum-free medium contains transferrin and other expensive animal-based-proteins, which is not favorable for the development of veterinary biological products. Therefore, it is necessary to develop a serum-free medium for the suspension culture of MDCK single cells, which is definite in components, convenient to prepare and use, lower in cost and suitable for producing the veterinary biological products in a large scale, so that the MDCK cells can be simply and rapidly educated from a serum adherent growth state to a serum-free suspension growth state, and a more advanced serum-free high-density suspension culture process for the MDCK cells is established.

### SUMMARY OF PRESENT INVENTION

In view of the deficiencies of the prior art, one of objectives of the present invention is to provide a serum-free medium for full suspension culture of MDCK cells. The medium supports the high-density full suspension culture of the MDCK single cells, greatly shortens the time for educating the MDCK cells from the serum adherent cultured cells to the serum-free full suspension cultured cells, and is applicable to the mass production of biological products and particularly the veterinary biological products.

In order to realize the above objective, the present invention adopts a technical solution as follows: a serum-free medium for full suspension culture of MDCK cells comprises components of the following concentrations:

basic metabolic nutrients:

| | | |
|---|---|---|
| D-glucose | 3000 to 10000 | mg/L; |
| sodium pyruvate | 50 to 600 | mg/L; |
| L-alanine | 5 to 30 | mg/L; |
| L-arginine | 150 to 600 | mg/L; |
| L-asparagine | 10 to 60 | mg/L; |
| L-aspartic acid | 10 to 60 | mg/L; |
| L-cystine | 10 to 80 | mg/L; |
| L-cysteine | 20 to 150 | mg/L; |
| L-glutamic acid | 5 to 60 | mg/L; |
| L-glutamine | 300 to 1500 | mg/L; |
| glycine | 10 to 100 | mg/L; |
| L-histidine | 10 to 150 | mg/L; |
| L-isoleucine | 20 to 150 | mg/L; |
| L-leucine | 50 to 250 | mg/L; |
| L-lysine | 50 to 150 | mg/L; |
| L-methionine | 20 to 150 | mg/L; |
| L-phenylalanine | 20 to 250 | mg/L; |
| L-proline | 20 to 200 | mg/L; |
| L-serine | 50 to 150 | mg/L; |
| L-threonine | 50 to 200 | mg/L; |
| L-tryptophan | 20 to 100 | mg/L; |
| L-tyrosine | 20 to 100 | mg/L; |
| L-valine | 50 to 200 | mg/L; |

nucleotide:

| | |
|---|---|
| hypoxanthine | 2 to 25 mg/L; |
| thymidine | 0.1 to 1 mg/L; |
| adenosine | 2 to 15 mg/L; |
| uridine | 2 to 15 mg/L; |
| cytidine | 2 to 15 mg/L; |
| guanosine | 2 to 15 mg/L; |

vitamins:

| | | |
|---|---|---|
| vitamin D | 0.01 to 0.20 | mg/L; |
| folic acid | 1 to 10 | mg/L; |
| nicotinamide | 1 to 10 | mg/L; |
| pyridoxine | 1 to 10 | mg/L; |
| thiamine | 1 to 10 | mg/L; |
| riboflavin | 0.1 to 1 | mg/L; |
| choline chloride | 10 to 50 | mg/L; |
| D-calcium pantothenate | 2 to 10 | mg/L; |
| inositol | 10 to 50 | mg/L; |

inorganic salts:

| | | |
|---|---|---|
| ferric nitrate | 10 to 50 | mg/L; |
| ferrous sulfate | 0.1 to 1 | mg/L; |
| magnesium sulfate | 20 to 100 | mg/L; |
| potassium chloride | 200 to 500 | mg/L; |
| sodium chloride | 5000 to 9000 | mg/L; |
| disodium hydrogen phosphate | 50 to 100 | mg/L; |
| sodium dihydrogen phosphate | 50 to 100 | mg/L; |
| sodium selenite | 20 to 100 | mg/L; |

shear force protective agent: 500 to 2500 mg/L;

cell clustering resisting agent: 20 to 150 mg/L;

pH buffer agent:

| | |
|---|---|
| sodium bicarbonate | 1000 to 3000 mg/L; |

pH indicator:

| | |
|---|---|
| phenol red | 5 to 15 mg/L; |

influenza virus proliferation

accelerant:

| | | |
|---|---|---|
| cholesterol | 1 to 10 | mg/L; |
| DL-α-tocopherol acetate | 0.3 to 3 | mg/L; |
| myristic acid | 0.05 to 0.5 | mg/L; |
| palmitic acid | 0.05 to 0.5 | mg/L; |
| stearic acid | 0.05 to 0.5 | mg/L; |
| magnesium chloride | 1000 to 5000 | mg/L; |
| calcium chloride | 50 to 250 | mg/L; |
| dimethyl sulfoxide | 2 to 20 | mg/L; |
| zinc sulfate | 0.2 to 2.0 | mg/L; |
| copper sulfate | 5 to 25 | mg/L; |
| manganese sulfate | 0.0001 to 0.001 | mg/L; |
| ammonium metavanadate | 0.001 to 0.005 | mg/L; |

other additives:

| | | |
|---|---|---|
| ammonium ferric citrate | 13.5 to 40.5 | mg/L; |
| insulin | 2 to 15 | mg/L; |
| soybean hydrolysate | 1000 to 5000 | mg/L; |
| ethanolamine | 1 to 10 | mg/L; |
| glutathione | 0.5 to 3 | mg/L. |

As a preferred embodiment of the present invention: in the serum-free medium for the full suspension culture of the MDCK cells, concentrations of various components are:

basic metabolic nutrients:

| | | |
|---|---|---|
| D-glucose | 4500 | mg/L; |
| sodium pyruvate | 220 | mg/L; |
| L-alanine | 22.3 | mg/L; |
| L-arginine | 273.9 | mg/L; |
| L-asparagine | 33.9 | mg/L; |
| L-aspartic acid | 33.3 | mg/L; |
| L-cystine | 42.67 | mg/L; |
| L-cysteine | 68.60 | mg/L; |
| L-glutamic acid | 36.8 | mg/L; |
| L-glutamine | 876 | mg/L; |
| glycine | 26.44 | mg/L; |
| L-histidine | 73.50 | mg/L; |
| L-isoleucine | 89.52 | mg/L; |
| Leucine | 159.38 | mg/L; |
| L-lysine | 107.21 | mg/L; |
| L-methionine | 87.74 | mg/L; |
| L-phenylalanine | 100.38 | mg/L; |
| L-proline | 96.47 | mg/L; |
| L-serine | 78.46 | mg/L; |
| L-threonine | 136.46 | mg/L; |
| L-tryptophan | 57.81 | mg/L; |
| L-tyrosine | 56.87 | mg/L; |
| L-valine | 96.85 | mg/L; |

nucleotide:

| | | |
|---|---|---|
| hypoxanthine | 10.3 | mg/L; |
| thymidine | 0.24 | mg/L; |
| adenosine | 7 | mg/L; |
| uridine | 7 | mg/L; |
| cytidine | 7 | mg/L; |
| guanosine | 7 | mg/L; |

vitamins:

| | |
|---|---|
| vitamin D | 0.072 mg/L; |
| folic acid | 5.32 mg/L; |
| nicotinamide | 3.14 mg/L; |
| pyridoxine | 3.15 mg/L; |
| thiamine | 3.23 mg/L; |
| riboflavin | 0.36 mg/L; |
| choline chloride | 26.01 mg/L; |
| D-calcium pantothenate | 5.82 mg/L; |
| inositol | 25 mg/L; |

inorganic salts:

| | | |
|---|---|---|
| ferric nitrate | 24.19 | mg/L; |
| ferrous sulfate | 0.417 | mg/L; |
| magnesium sulfate | 48.8 | mg/L; |
| potassium chloride | 311.8 | mg/L; |
| sodium chloride | 6860 | mg/L; |
| disodium hydrogen phosphate | 71 | mg/L; |
| sodium dihydrogen phosphate | 62.5 | mg/L; |
| sodium selenite | 51.88 | mg/L; |

shear force protective agent: 1600 mg/L;

cell clustering resisting agent: 50 mg/L;

pH buffer agent:

| | |
|---|---|
| sodium bicarbonate | 2200 mg/L; |

pH indicator:

| | |
|---|---|
| phenol red | 8 mg/L; |

influenza virus proliferation

accelerant:

| | | |
|---|---|---|
| cholesterol | 3.13 | mg/L; |
| DL-α-tocopherol acetate | 1.39 | mg/L; |
| myristic acid | 0.2284 | mg/L; |
| palmitic acid | 0.256 | mg/L; |
| stearic acid | 0.285 | mg/L; |
| magnesium chloride | 2856 | mg/L; |
| calcium chloride | 174.9 | mg/L; |
| dimethyl sulfoxide | 11 | mg/L; |
| zinc sulfate | 0.8 | mg/L; |
| copper sulfate | 15.97 | mg/L; |
| manganese sulfate | 0.000302 | mg/L; |
| ammonium metavanadate | 0.00234 | mg/L; |

other additives:

| | | |
|---|---|---|
| ammonium ferric citrate | 27 | mg/L; |
| insulin | 6.94 | mg/L; |
| soybean hydrolysate | 2100 | mg/L; |
| ethanolamine | 3.46 | mg/L; |
| glutathione | 1.4 | mg/L. |

As a preferred embodiment of the present invention, the shear force protective agent is segmented polyether F68,

As a preferred embodiment of the present invention, the cell clustering resisting agent is dextran sulfate.

Another objective of the present invention is to provide a preparation method of a serum-free medium for full suspension culture of MDCK cells. The method is simple, rapid and high in efficiency and facilitates the mass production.

In order to realize the above objective, the present invention adopts a technical solution as follows: a preparation method of a serum-free medium for full suspension culture of MDCK cells comprises the following steps:

1) preparing a mixed solution: dissolving and mixing raw materials in one of the following methods:

I) mixing the raw materials and grinding them into fine powder, and then dissolving the obtained fine powder in a solvent at 10 to 30° C. to obtain a mixed solution;

II) respectively dissolving raw materials in the solvent to obtain a raw material solution; and mixing the obtained raw material solutions at a temperature of 10 to 30° C. to obtain a mixed solution; and

2) regulating pH: regulating the pH of the mixed solution to 6.3 to 6.7, and setting a constant volume to obtain the serum-free medium for the full suspension culture of the MDCK cells.

As a preferred embodiment of the present invention, in step 1 the solvent, is pyrogen-free ultra-pure water.

As a preferred embodiment of the present invention, in step 2), sodium hydroxide is added to regulate the pH value of the obtained mixed solution.

The present invention has the beneficial effects as follows:

1 The serum-free medium for the full suspension culture of the MDCK cells contains no animal serum, is low in cost, supports the high-density full suspension culture of the MDCK single cells, and is definite in components, easy to prepare and convenient to use;

2. the medium of the present invention effectively, shortens the time for educating the MDCK cells from the serum adherent cultured cells to the serum-free full-suspension cultured cells, increases the production efficiency and obtains high-quality full suspension cells; and

3 the preparation method of the present invention is simple, fast and high in efficiency and facilitates the mass production.

### DESCRIPTION OF THE DRAWINGS

FIG. 1 is a curve chart of living cell density and cell activity in embodiment 4;

FIG. 2 is a graph of morphology of MDCK cells in a serum adherent culture state in embodiment 5;

FIG. 3 is a graph of morphology of MDCK cells educated with a serum-free medium of the present invention in embodiment 5;

FIG. 4 is a graph of morphology of suspension cultured MDCKS cells obtained by employing a serum-free medium SFM4 Mega Vir of Hyclone Company in a direct education method in embodiment 5; and

FIG. 5 is a graph of morphology of MDCK.SUS2 cells obtained by employing a commercial serum-free medium SMIF8 developed by Gibco Company in an indirect education method in embodiment 5.

### DETAILED DESCRIPTION OF PREFERRED EMBODIMENTS

The present invention is further described below in combination with specific implementation manners:

Specific implementation manners:

A serum-free medium for full suspension culture of MDCK cells comprises components of the following concentrations:

basic metabolic nutrients:

| | | |
|---|---|---|
| D-glucose | 3000 to 10000 | mg/L; |
| sodium pyruvate | 50 to 600 | mg/L; |
| L-alanine | 5 to 30 | mg/L; |
| L-arginine | 150 to 600 | mg/L; |
| L-asparagine | 10 to 60 | mg/L; |
| L-aspartic acid | 10 to 60 | mg/L; |
| L-cystine | 10 to 80 | mg/L; |
| L-cysteine | 20 to 150 | mg/L; |
| L-glutamic acid | 5 to 60 | mg/L; |
| L-glutamine | 300 to 1500 | mg/L; |
| glycine | 10 to 100 | mg/L; |
| L-histidine | 10 to 150 | mg/L; |
| L-isoleucine | 20 to 150 | mg/L; |
| L-leucine | 50 to 250 | mg/L; |
| L-lysine | 50 to 150 | mg/L; |
| L-methionine | 20 to 150 | mg/L; |
| L-phenylalanine | 20 to 250 | mg/L; |
| L-proline | 20 to 200 | mg/L; |
| L-serine | 50 to 150 | mg/L; |
| L-threonine | 50 to 200 | mg/L; |
| L-tryptophan | 20 to 100 | mg/L; |
| L-tyrosine | 20 to 100 | mg/L; |
| L-valine | 50 to 200 | mg/L; |

nucleotide:

| | |
|---|---|
| hypoxanthine | 2 to 25 mg/L; |
| thymidine | 0.1 to 1 mg/L; |
| adenosine | 2 to 15 mg/L; |
| uridine | 2 to 15 mg/L; |
| cytidine | 2 to 15 mg/L; |
| guanosine | 2 to 15 mg/L; |

vitamins:

| | | |
|---|---|---|
| vitamin D | 0.01 to 0.20 | mg/L; |
| folic acid | 1 to 10 | mg/L; |
| nicotinamide | 1 to 10 | mg/L; |
| pyridoxine | 1 to 10 | mg/L; |
| thiamine | 1 to 10 | mg/L; |
| riboflavin | 0.1 to 1 | mg/L; |
| choline chloride | 10 to 50 | mg/L; |
| D-calcium pantothenate | 2 to 10 | mg/L; |
| inositol | 10 to 50 | mg/L; |

inorganic salts:

| | | |
|---|---|---|
| ferric nitrate | 10 to 50 | mg/L; |
| ferrous sulfate | 0.1 to 1 | mg/L; |
| magnesium sulfate | 20 to 100 | mg/L; |
| potassium chloride | 200 to 500 | mg/L; |
| sodium chloride | 5000 to 9000 | mg/L; |
| disodium hydrogen phosphate | 50 to 100 | mg/L; |
| sodium dihydrogen phosphate | 50 to 100 | mg/L; |
| sodium selenite | 20 to 100 | mg/L; |

shear force protective agent:

| | |
|---|---|
| segmented polyether F68 | 500 to 2500 mg/L; |

cell clustering resisting agent:

| | |
|---|---|
| dextran sulfate | 20 to 150 mg/L; |

pH buffer agent:

| | |
|---|---|
| sodium bicarbonate | 1000 to 3000 mg/L; |

pH indicator:

| | |
|---|---|
| phenol red | 5 to 15 mg/L; |

influenza virus proliferation accelerant:

| | | |
|---|---|---|
| cholesterol | 1 to 10 | mg/L; |
| DL-α-tocopherol acetate | 0.3 to 3 | mg/L; |
| myristic acid | 0.05 to 0.5 | mg/L; |
| palmitic acid | 0.05 to 0.5 | mg/L; |
| stearic acid | 0.05 to 0.5 | mg/L; |
| magnesium chloride | 1000 to 5000 | mg/L; |
| calcium chloride | 50 to 250 | mg/L; |
| dimethyl sulfoxide | 2 to 20 | mg/L; |
| zinc sulfate | 0.2 to 2.0 | mg/L; |
| copper sulfate | 5 to 25 | mg/L; |
| manganese sulfate | 0.0001 to 0.001 | mg/L; |
| ammonium metavanadate | 0.001 to 0.005 | mg/L; |

other additives:

| | | |
|---|---|---|
| ammonium ferric citrate | 13.5 to 40.5 | mg/L; |
| insulin | 2 to 15 | mg/L; |
| soybean hydrolysate | 1000 to 5000 | mg/L; |
| ethanolamine | 1 to 10 | mg/L; |
| glutathione | 0.5 to 3 | mg/L. |

The hypoxanthine and the thymidine are selected, in the nucleotide, so that the nucleotide synthesis of the MDCK cells can be promoted, and the, growth of the cells can be ensured; and if the proportion of the hypoxanthine and the thymidine is excessively high, the growth of the cells may be inhibited.

Ammonium ferric citrate is selected in other additives and used to substitute transferrin to play the original effect, the growth of the cells and the iron metabolism are not affected, animal protein components in the serum-free medium can be reduced, and the cost of the medium and the uncertainty and unsafety in production can be reduced; the ammonium ferric citrate absorbs iron through a divalent metal ion channel DMT1, while the transferrin absorbs the iron through a transferrin receptor, so that compared with the transferrin, the ammonium ferric citrate increases the absorption rate of the iron in the MDCK cells; if the concentration of the ammonium ferric citrate is excessively high, the growth of the MDCK cells may be inhibited; and if the concentration is excessively low, the MDCK cells are insufficient to absorb the iron.

The concentration of insulin in the other additives is 2 to 15 mg/L, so that the metabolism of the glucose can be promoted, the growth of the MDCK cells can be ensured and the activity of the MDCK cells can be maintained.

The concentration of the soybean hydrolysate in the other additives is 1000 to 5000 mg/mL, so that the supply of other auxiliary factors such as the vitamins, metal ions, amino acid and the like can be ensured, and the absorption of the amino acid in the MDCK cells can be improved.

A preparation method of a serum-free medium for full suspension culture of MDCK cells comprises the following steps:

1) a mixed solution is prepared: raw materials are dissolved end mixed in one of the following methods:

I) raw materials are mixed and then ground into fine powder, and the obtained fine powder is dissolved in pyrogen-free ultra-pure water at 10 to 30° C. to obtain a mixed solution;

II) raw materials are respectively dissolved in the pyrogen-free ultra-pure water to obtain, a raw material solution; and then the obtained raw material solutions are mixed at the temperature of 10 to 30° C. to obtain a mixed solution; and

2) pH is regulated: the sodium hydroxide is added to regulate the pH of the mixed solution to 6.3 to 6.7, and after a constant volume is set, the serum-free medium for the full suspension culture of the MDCK cells is obtained.

Specific embodiments

### Embodiment 1

The present embodiment discloses a serum-free medium for full suspension culture of MDCK cells. The serum-free medium for the full suspension culture of the MDCK cells comprises components of the following concentrations: basic metabolic nutrients:

| | | |
|---|---|---|
| D-glucose | 3715 | mg/L; |
| sodium pyruvate | 110 | mg/L; |
| L-alanine | 11.2 | mg/L; |
| L-arginine | 219.1 | mg/L; |
| L-asparagine | 22.6 | mg/L; |
| L-aspartic acid | 22.2 | mg/L; |
| L-cystine | 28.45 | mg/L; |
| L-cysteine | 54.88 | mg/L; |
| L-glutamic acid | 18.4 | mg/L; |
| L-glutamine | 584 | mg/L; |
| glycine | 13.22 | mg/L; |
| L-histidine | 36.75 | mg/L; |
| L-isoleucine | 44.76 | mg/L; |
| L-leucine | 79.69 | mg/L; |
| L-lysine | 85.77 | mg/L; |
| L-methionine | 43.87 | mg/L; |
| L-phenylalanine | 50.19 | mg/L; |
| L-proline | 48.24 | mg/L; |
| L-serine | 62.77 | mg/L; |
| L-threonine | 85.29 | mg/L; |
| L-tryptophan | 38.54 | mg/L; |
| L-tyrosine | 45.50 | mg/L; |
| L-valine | 58.11 | mg/L; |

nucleotide:

| | |
|---|---|
| hypoxanthine | 5.2 mg/L; |
| thymidine | 0.12 mg/L; |
| adenosine | 3.5 mg/L; |
| uridine | 3.5 mg/L; |
| cytidine | 3.5 mg/L; |
| guanosine | 3.5 mg/L; |

vitamins:

| | | |
|---|---|---|
| vitamin D | 0.036 | mg/L; |
| folic acid | 2.66 | mg/L; |
| nicotinamide | 1.51 | mg/L; |
| pyridoxine | 1.6 | mg/L; |
| thiamine | 1.62 | mg/L; |
| riboflavin | 0.18 | mg/L; |
| choline chloride | 13 | mg/L; |
| D-calcium pantothenate | 2.91 | mg/L; |
| inositol | 12.5 | mg/L; |

inorganic salts:

| | | |
|---|---|---|
| ferric nitrate | 24.19 | mg/L; |
| ferrous sulfate | 0.417 | mg/L; |
| magnesium sulfate | 24.4 | mg/L; |
| potassium chloride | 311.8 | mg/L; |
| sodium chloride | 5488 | mg/L; |
| disodium hydrogen phosphate | 71 | mg/L; |
| sodium dihydrogen phosphate | 62.5 | mg/L; |
| sodium selenite | 25.94 | mg/L; |

shear force protective agent:

| | |
|---|---|
| segmented polyether F68 | 1000 mg/L; |

cell clustering resisting agent:

| | |
|---|---|
| dextran sulfate | 25 mg/L; |

pH buffer agent:

| | |
|---|---|
| sodium bicarbonate | 2200 mg/L; |

pH indicator:

| | |
|---|---|
| phenol red | 8 mg/L; |

influenza virus proliferation

accelerant:

| | | |
|---|---|---|
| cholesterol | 1.57 | mg/L; |
| DL-α-tocopherol acetate | 0.7 | mg/L; |
| myristic acid | 0.1142 | mg/L; |
| palmitic acid | 0.128 | mg/L; |
| stearic acid | 0.143 | mg/L; |
| magnesium chloride | 1904 | mg/L; |
| calcium chloride | 116.6 | mg/L; |
| dimethyl sulfoxide | 5.5 | mg/L; |
| zinc sulfate | 0.4 | mg/L; |
| copper sulfate | 7.98 | mg/L; |
| manganese sulfate | 0.000151 | mg/L; |
| ammonium metavanadate | 0.00117 | mg/L; |

other additives:

| | | |
|---|---|---|
| ammonium ferric citrate | 13.5 | mg/L; |
| insulin | 5 | mg/L; |
| soybean hydrolysate | 1400 | mg/L; |
| ethanolamine | 1.73 | mg/L; |
| glutathione | 0.7 | mg/L. |

The serum-free medium for the full suspension culture of MDCK cells is prepared according to the following steps:

1) the raw materials are mixed and then ground into fine powder, the obtained fine powder is dissolved in pyrogen-free ultra-pure water at 10 to 30° C., the concentration of each raw material is as described above, and the mixed solution is obtained;

2) the sodium hydroxide is added to regulate the pH of the mixed solution to 6.4, and after the constant volume is set, the serum-free medium DHN-1 for the full suspension culture of the MDCK cells is obtained.

### Embodiment 2

According to the present embodiment, the serum-free medium for the full suspension culture of the MDCK cells comprises components of the following concentrations

basic metabolic nutrients:

| | | |
|---|---|---|
| D-glucose | 4500 | mg/L; |
| sodium pyruvate | 220 | mg/L; |
| L-alanine | 22.3 | mg/L; |
| L-arginine | 273.9 | mg/L; |
| L-asparagine | 33.9 | mg/L; |
| L-aspartic acid | 33.3 | mg/L; |
| L-cystine | 42.67 | mg/L; |
| L-cysteine | 68.60 | mg/L; |
| L-glutamic acid | 36.8 | mg/L; |
| L-glutamine | 876 | mg/L; |
| glycine | 26.44 | mg/L; |
| L-histidine | 73.50 | mg/L; |
| L-isoleucine | 89.52 | mg/L; |
| L-leucine | 159.38 | mg/L; |
| L-lysine | 107.21 | mg/L; |
| L-methionine | 87.74 | mg/L; |
| L-phenylalanine | 100.38 | mg/L; |
| L-proline | 96.47 | mg/L; |
| L-serine | 78.46 | mg/L; |
| L-threonine | 136.46 | mg/L; |
| L-tryptophan | 57.81 | mg/L; |
| L-tyrosine | 56.87 | mg/L; |
| L-valine | 96.85 | mg/L; |

nucleotide:

| | | |
|---|---|---|
| hypoxanthine | 10.3 | mg/L; |
| thymidine | 0.24 | mg/L; |
| adenosine | 7 | mg/L; |
| uridine | 7 | mg/L; |
| cytidine | 7 | mg/L; |
| guanosine | 7 | mg/L; |

vitamins:

| | | |
|---|---|---|
| vitamin D | 0.072 | mg/L; |
| folic acid | 5.32 | mg/L; |
| nicotinamide | 3.14 | mg/L; |
| pyridoxine | 3.15 | mg/L; |
| thiamine | 3.23 | mg/L; |
| riboflavin | 0.36 | mg/L; |
| choline chloride | 26.01 | mg/L; |
| D-calcium pantothenate | 5.82 | mg/L; |
| inositol | 25 | mg/L; |

inorganic salts:

| | | |
|---|---|---|
| ferric nitrate | 24.19 | mg/L; |
| ferrous sulfate | 0.417 | mg/L; |
| magnesium sulfate | 48.8 | mg/L; |
| potassium chloride | 311.8 | mg/L; |
| sodium chloride | 6860 | mg/L; |
| disodium hydrogen phosphate | 71 | mg/L; |
| sodium dihydrogen phosphate | 62.5 | mg/L; |
| sodium selenite | 51.88 | mg/L; |

shear force protective agent:

| | |
|---|---|
| segmented polyether F68 | 1600 mg/L; |

cell clustering resisting agent:

| | |
|---|---|
| dextran sulfate | 50 mg/L; |

pH buffer agent:

| | |
|---|---|
| sodium bicarbonate | 2200 mg/L; |

pH indicator:

| | |
|---|---|
| phenol red | 8 mg/L; |

influenza virus proliferation accelerant:

| | | |
|---|---|---|
| cholesterol | 3.13 | mg/L; |
| DL-α-tocopherol acetate | 1.39 | mg/L; |
| myristic acid | 0.2284 | mg/L; |
| palmitic acid | 0.256 | mg/L; |
| stearic acid | 0.285 | mg/L; |
| magnesium chloride | 2856 | mg/L; |
| calcium chloride | 174.9 | mg/L; |
| dimethyl sulfoxide | 11 | mg/L; |
| zinc sulfate | 0.8 | mg/L; |
| copper sulfate | 15.97 | mg/L; |
| manganese sulfate | 0.000302 | mg/L; |
| ammonium metavanadate | 0.00234 | mg/L; |

other additives:

| | | |
|---|---|---|
| ammonium ferric citrate | 27 | mg/L; |
| insulin | 6.94 | mg/L; |
| soybean hydrolysate | 2100 | mg/L; |
| ethanolamine | 3.46 | mg/L; |
| glutathione | 1.4 | mg/L. |

The serum-free medium for the full suspensor culture of the MDCK cells is prepared according to the following steps:

1 ) the raw materials are mixed and then ground into fine powder, the obtained fine powder is dissolved in pyrogen-free ultra-pure water at 10 to 30° C., the concentration of each raw material is as described above, and the mixed solution is obtained: and

2) the sodium hydroxide is added to regulate the pH of the mixed solution to 6.5, and after the constant volume is set, the serum-free medium DHN-2 for the full suspension culture of the MDCK cells is obtained.

### Embodiment 3

According to the present embodiment, the serum-free medium for the full suspension culture of the MDCK cells comprises components of the following concentrations:

basic metabolic nutrients:

| | | |
|---|---|---|
| D-glucose | 9000 | mg/L; |
| sodium pyruvate | 440 | mg/L; |
| L-alanine | 22.5 | mg/L; |
| L-arginine | 547.8 | mg/L; |
| L-asparagine | 45.2 | mg/L; |
| L-aspartic acid | 55.5 | mg/L; |
| L-cystine | 64.01 | mg/L; |
| L-cysteine | 102.9 | mg/L; |
| L-glutamic acid | 55.2 | mg/L; |
| L-glutamine | 1460 | mg/L; |
| glycine | 52.08 | mg/L; |
| L-histidine | 110.25 | mg/L; |
| L-isoleucine | 134.28 | mg/L; |
| L-leucine | 239.07 | mg/L; |
| L-lysine | 134.01 | mg/L; |
| L-methionine | 109.68 | mg/L; |
| L-phenylalanine | 200.76 | mg/L; |
| L-proline | 144.71 | mg/L; |
| L-serine | 117.69 | mg/L; |
| L-threonine | 170.57 | mg/L; |
| L-tryptophan | 77.07 | mg/L; |
| L-tyrosine | 85.31 | mg/L; |
| L-valine | 145.28 | mg/L; |

nucleotide:

| | |
|---|---|
| hypoxanthine | 20.6 mg/L; |
| thymidine | 0.48 mg/L; |
| adenosine | 10.5 mg/L; |
| uridine | 10.5 mg/L; |
| cytidine | 10.5 mg/L; |
| guanosine | 10.5 mg/L; |

vitamins:

| | | |
|---|---|---|
| vitamin D | 0.144 | mg/L; |
| folic acid | 7.98 | mg/L; |
| nicotinamide | 6.28 | mg/L; |
| pyridoxine | 6.3 | mg/L; |
| thiamine | 6.46 | mg/L; |
| riboflavin | 0.72 | mg/L; |
| choline chloride | 39.02 | mg/L; |
| D-calcium pantothenate | 8.73 | mg/L; |
| inositol | 37.5 | mg/L; |

inorganic salts:

| | | |
|---|---|---|
| ferric nitrate | 24.19 | mg/L; |
| ferrous sulfate | 0.417 | mg/L; |
| magnesium sulfate | 73.2 | mg/L; |
| potassium chloride | 311.8 | mg/L; |
| sodium chloride | 8575 | mg/L; |
| disodium hydrogen | 71 | mg/L; |
| phosphate | | |
| sodium dihydrogen | 62.5 | mg/L; |
| phosphate | | |
| sodium selenite | 77.82 | mg/L; |

shear force protective agent:

| | |
|---|---|
| segmented polyether F68 | 2200 mg/L; |

cell clustering resisting agent:

| | |
|---|---|
| dextran sulfate | 100 mg/L; |

pH buffer agent:

| | |
|---|---|
| sodium bicarbonate | 2200 mg/L; |

pH indicator:

| | |
|---|---|
| phenol red | 8 mg/L; |

influenza virus proliferation accelerant:

| | | |
|---|---|---|
| cholesterol | 6.26 | mg/L; |
| DL-α-tocopherol acetate | 2.1 | mg/L; |
| myristic acid | 0.3426 | mg/L; |
| palmitic acid | 0.384 | mg/L; |
| stearic acid | 0.428 | mg/L; |
| magnesium chloride | 4762 | mg/L; |
| calcium chloride | 233.2 | mg/L; |
| dimethyl sulfoxide | 16.5 | mg/L; |
| zinc sulfate | 1.6 | mg/L; |
| copper sulfate | 23.96 | mg/L; |
| manganese sulfate | 0.000453 | mg/L; |
| ammonium metavanadate | 0.00351 | mg/L; |

other additives:

| | | |
|---|---|---|
| ammonium ferric citrate | 40.5 | mg/L; |
| insulin | 10.41 | mg/L; |
| soybean hydrolysate | 4200 | mg/L; |
| ethanolamine | 5.19 | mg/L; |
| glutathione | 2.1 | mg/L. |

The serum-free medium for the full suspension culture of the MDCK cells is prepared according to the following, steps:

1) the raw materials are mixed and then ground into fine powder, the obtained fine powder is dissolved in pyrogen-free ultra-pure water at 10 to 30° C., the concentration of each raw material is as described above, and the mixed solution is obtained;

2) the sodium hydroxide is added to regulate the pH of the mixed solution to 6.7, and after the constant'volume is set, the serum-free medium DHN-3 for the full suspension culture of the MDCK cells is obtained.

A characteristic test is carried out for the medium obtained in embodiments 1 to 3:

1. Instrument: bio-reactor Bio-Bundle purchased from Holland Applikon Biotechnology Company), and a volume of a tank body is 3L;

2. Cells: MDCK cell lines suitable for the serum-free full suspension culture, provided by East China University of Science and Technology;

3. Serum-free medium for reference: commercial serum-free medium SFM4 Mega Vir (purchased from Hyclone Company);

4. Culture method: the cells are inoculated into the bio-reactor at a cell density of 0.5×10⁶ cells/mL and subjected to mass culture under the conditions of 37° C. and 5% CO₂, the cells are sampled every 24 h for counting the living cells, and the growth rate of the cells is calculated. Results are shown in Table 1 and Table 2:

TABLE 1
Living cell density at different time (10⁶ cells/mL)

Medium

| | SFM4 Mega | | | |
|---|---|---|---|---|
| | Vir | | | |
| Time | (Reference) | DHN-1 | DHN-2 | DHN-3 |
| 0 | 0.5 | 0.5 | 0.5 | 0.5 |
| 24 | 0.86 | 1.08 | 1.25 | 1.18 |
| 48 | 1.54 | 2.87 | 3.22 | 3.16 |
| 72 | 3.58 | 6.53 | 7.05 | 6.18 |
| 96 | 2.67 | 5.87 | 5.90 | 5.45 |

TABLE 2
Cell growth rate and doubling time

| | Medium | | | |
|---|---|---|---|---|
| | SFM4 Mega | | | |
| | Vir | | | |
| Item | (Reference) | DHN-1 | DHN-2 | DHN-3 |
| Average specific growth | 0.57 | 0.80 | 0.91 | 0.77 |
| rate of cells at a | | | | |
| non-exponential growth | | | | |
| period (d⁻¹) | | | | |
| Average doubling time of | 0.79 | 0.39 | 0.32 | 0.43 |
| cells at a non-exponential | | | | |
| growth period (d) | | | | |

Compared with the commercial serum-free medium SFM4 Mega Vir of a control group for the suspension culture of the MDCK cells, by adopting the serum-free medium provided by the present invention, the supported living cell density in the culture process is greatly increased; and furthermore, the specific, growth rate of the cells at the non-exponential growth period is increased from the maximum 0.57 d-1 of the control group to 0.91 d-1 in DHN-2 of the embodiment 2, and the doubling time of the cells is shortened from the maximum 0.79 d of the control group to 0.32 d in DHN-2 of the embodiment 2. It can be seen that by adopting the serum-free medium of the present invention to culture the MDCK cells, both the cell growth rate and the cell activity are greatly improved.

### Embodiment 4

The serum-free medium DHN-2 prepared in embodiment 2 is used to perform the serum-free full suspension culture education for the serum adherent cultured MDCK cells. The cell education process is as follows:

1) when the adherent MDCK cells cultured by DMEM containing 10% of new-born calf serum are cultured to the cell confluence of 80% to 90%, the original medium is abandoned, the cell layers are washed twice with pancreatin so as to neutralize the residual serum, and liquid is abandoned; a pancreatin solution is continuously added to cover the MDCK cells to perform the digestion for 5 to 15 min; after all cells become round, the digestion was terminated by adding medium containing 10% of fetal bovine serum with the volume four times of the volume of a digestive solution; and the cells are blown and beaten by using a pipette, the cells are suspended, a cell suspension solution is collected and centrifuged at 1000 rpm for 5 min, then supernatant is abandoned, and cell clusters are obtained;

2) the cell clusters are re-suspended by using the serum-free medium DHN-2 until the cell density is about 1.5*10⁶ cells/mL, and a cell re-suspension solution is obtained;

3) the cell re-suspension solution is added into a square vase and cultured in an incubator at :a rotation speed of 30 rpm, a temperature of 37° C., and 5% of CO₂; after two-generation culture, the cultured cell re-suspension solution is transferred into a 125 mL of shake flask, and the rotation speed is increased to 120 rpm. The cells are sampled every 24 h, the sampled cells are counted and subjected to the activity analysis, the cell density is diluted with fresh medium DHN-2 to about 1.5*10⁶ cells/mL every 48 h, subculture is coontinued on a shaking table, and the MDCK cells suitable for the serum-free full suspension culture are obtained, and

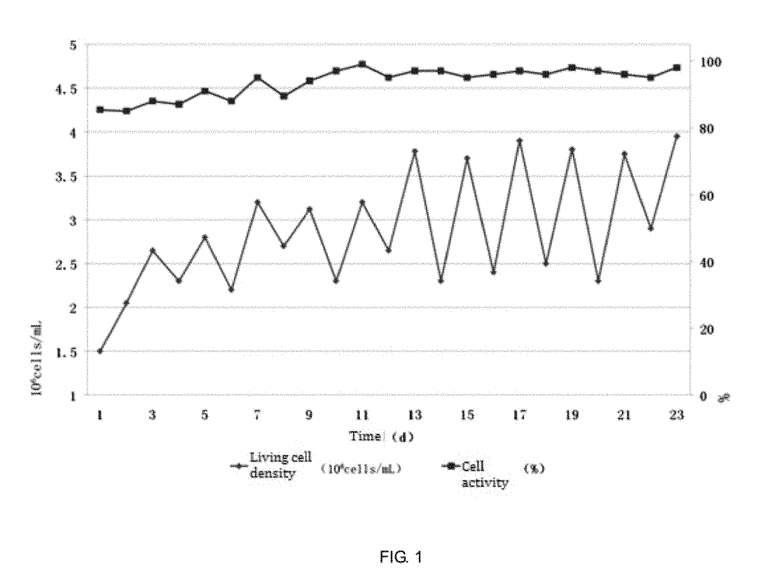

4) the living cell density and the cell activity are shown in FIG. 1: after the MDCK adherent cells are educated for 6 generations (13th day after the domestication) in the serum-free medium DHN-2, the cell growth is gradually stable, and the cell activity is kept at 95% or higher. Thus, it can prove that in the serum-free medium of the present invention, the MDCK adherent cells can be suitable for the suspension culture and grow stably only in two weeks, thereby greatly shortening the time for educating the MDCK cells from the adherent cells to the serum-free full suspension cells.

### Embodiment 5

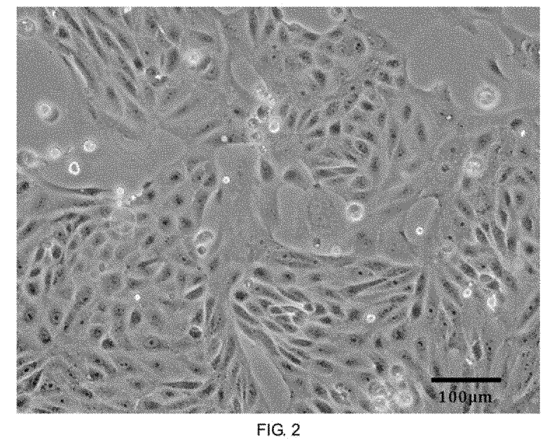

The morphology of the serum-free full suspension cultured MDCK cells educated with the medium of the present invention is compared with the morphology of the adherent culture cells and the cells cultured with other serum-free media, and results are shown in FIG. 2 to FIG. 5:

In FIG. 2, the MDCK cells in a serum adherent culture state are attached onto the surface of the medium and present a paving stone shape.

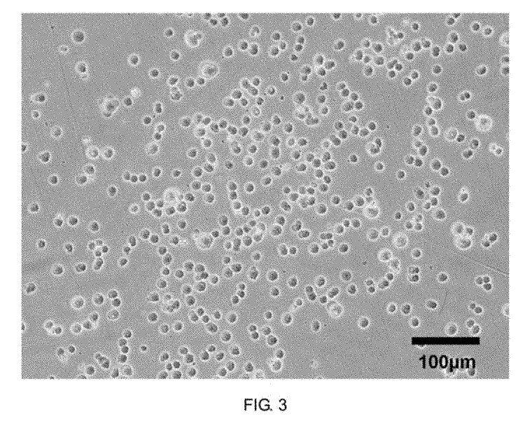

FIG. 3 illustrates the morphology of the full suspension cultured MDCK cells educated with the serum-free medium of the present invention, the cells present an individual scattering shape and have no clustering phenomenon, the cell morphology is complete, the boundary is smooth and clear, and the size is uniform.

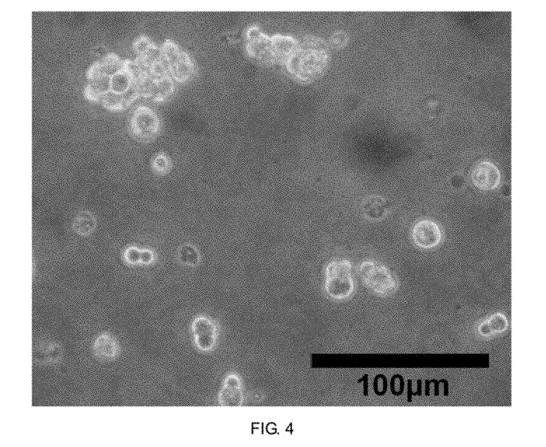

FIG. 4 shows the suspension cultured MDCKS cells obtained by employing the serum-free medium SFM4 Mega Vir of Hyclone Company in a direct education method, and the picture is from Zhang Liangyan, Yao Zhidong et al. “Suspension Education and Primary Application of MDCK Cells”, biological technological communication, 2013, 24(3): 382-384, and it can be seen from the picture that a plurality of cells are clustered, individual cells are rare, and the cells are non-uniform in size.

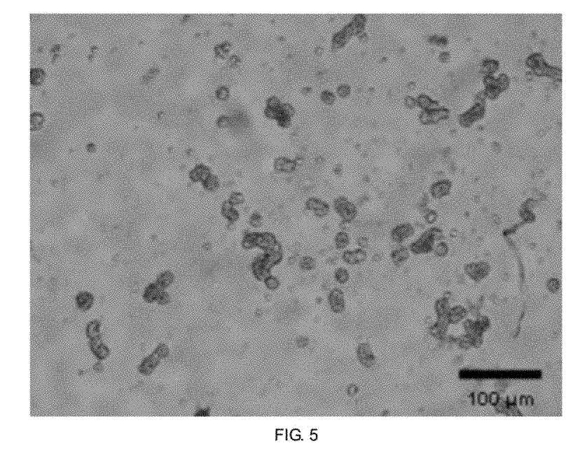

FIG. 5 shows the MDCK.SUS2 cells obtained by employing the commercial serum-free medium SMIF8 developed by Gibco Company in an indirect education method; the picture is from: V. Lohr, Y. Genzel, et at. “A new MDCK suspension line cultivated in a fully defined medium in stirred-tank and wave bioreactor”. Vaccine.2010,28(3):6256-6264; and it can be seen from the picture that the morphology of the cells when in the suspension culture in the serum-free medium is also in a clustered shape, but the cluster is small, and the cells are non-uniform in size and bad in state.

Therefore, the adherent cultured MDCK cells are educated to the suspension culture state in the serum-free medium of the present invention, the cells grow in an individually scattering manner, the cell morphology is full and the size is uniform; and the cell quality is high.

It will be apparent to those skilled in the art that various other corresponding changes and variations may be made in accordance with the technical solutions and concepts described above, and all of the changes and variations shall belong to the protection scope of the claims of the present invention.

We claim:

1. A serum-free medium for full suspension culture of MDCK cells, comprising components of the following concentrations:
basic metabolic nutrients:

| | | |
|---|---|---|
| D-glucose | 3000 to 10000 | mg/L; |
| sodium pyruvate | 50 to 600 | mg/L; |
| L-alanine | 5 to 30 | mg/L; |
| L-arginine | 150 to 600 | mg/L; |
| L-asparagine | 10 to 60 | mg/L; |
| L-aspartic acid | 10 to 60 | mg/L; |
| L-cystine | 10 to 80 | mg/L; |
| L-cysteine | 20 to 150 | mg/L; |
| L-glutamic acid | 5 to 60 | mg/L; |
| L-glutamine | 300 to 1500 | mg/L; |
| glycine | 10 to 100 | mg/L; |
| L-histidine | 10 to 150 | mg/L; |
| L-isoleucine | 20 to 150 | mg/L; |
| L-leucine | 50 to 250 | mg/L; |
| L-lysine | 50 to 150 | mg/L; |
| L-methionine | 20 to 150 | mg/L; |
| L-phenylalanine | 20 to 250 | mg/L; |
| L-proline | 20 to 200 | mg/L; |
| L-serine | 50 to 150 | mg/L; |
| L-threonine | 50 to 200 | mg/L; |
| L-tryptophan | 20 to 100 | mg/L; |
| L-tyrosine | 20 to 100 | mg/L; |
| L-valine | 50 to 200 | mg/L; |

nucleotide:

| | |
|---|---|
| hypoxanthine | 2 to 25 mg/L; |
| thymidine | 0.1 to 1 mg/L; |
| adenosine | 2 to 15 mg/L; |
| uridine | 2 to 15 mg/L; |
| cytidine | 2 to 15 mg/L; |
| guanosine | 2 to 15 mg/L; |

vitamins:

| | | |
|---|---|---|
| vitamin D | 0.01 to 0.20 | mg/L; |
| folic acid | 1 to 10 | mg/L; |
| nicotinamide | 1 to 10 | mg/L; |
| pyridoxine | 1 to 10 | mg/L; |
| thiamine | 1 to 10 | mg/L; |
| riboflavin | 0.1 to 1 | mg/L; |
| choline chloride | 10 to 50 | mg/L; |
| D-calcium pantothenate | 2 to 10 | mg/L; |
| inositol | 10 to 50 | mg/L; |

inorganic salts:

| | | |
|---|---|---|
| ferric nitrate | 10 to 50 | mg/L; |
| ferrous sulfate | 0.1 to 1 | mg/L; |
| magnesium sulfate | 20 to 100 | mg/L; |
| potassium chloride | 200 to 500 | mg/L; |
| sodium chloride | 5000 to 9000 | mg/L; |
| disodium hydrogen phosphate | 50 to 100 | mg/L; |
| sodium dihydrogen phosphate | 50 to 100 | mg/L; |
| sodium selenite | 20 to 100 | mg/L; |

shear force protective agent: 500 to 2500 mg/L;
cell clustering resisting agent 20 to 150 mg/L;
pH buffer agent:

| | |
|---|---|
| sodium bicarbonate | 1000 to 3000 mg/L; |

pH indicator:

| | |
|---|---|
| phenol red | 5 to 15 mg/L; |

influenza virus proliferation
accelerant:

| | | |
|---|---|---|
| cholesterol | 1 to 10 | mg/L; |
| DL-α-tocopherol acetate | 0.3 to 3 | mg/L; |
| myristic acid | 0.05 to 0.5 | mg/L; |
| palmitic acid | 0.05 to 0.5 | mg/L; |
| stearic acid | 0.05 to 0.5 | mg/L; |
| magnesium chloride | 1000 to 5000 | mg/L; |
| calcium chloride | 50 to 250 | mg/L; |
| dimethyl sulfoxide | 2 to 20 | mg/L; |
| zinc sulfate | 0.2 to 2.0 | mg/L; |
| copper sulfate | 5 to 25 | mg/L; |
| manganese sulfate | 0.0001 to 0.001 | mg/L; |
| ammonium metavanadate | 0.001 to 0.005 | mg/L; |

other additives:

| | | |
|---|---|---|
| ammonium ferric citrate | 13.5 to 40.5 | mg/L; |
| insulin | 2 to 15 | mg/L; |
| soybean hydrolysate | 1000 to 5000 | mg/L; |
| ethanolamine | 1 to 10 | mg/L; |
| glutathione | 0.5 to 3 | mg/L. |

2. The serum-free medium for the full suspension culture of the MDCK cells of claim 1, wherein the serum-free medium for the full suspension culture of the MDCK cells comprises components of the following concentrations:
basic metabolic nutrients:

| | | |
|---|---|---|
| D-glucose | 4500 | mg/L; |
| sodium pyruvate | 220 | mg/L; |
| L-alanine | 22.3 | mg/L; |
| L-arginine | 273.9 | mg/L; |
| L-asparagine | 33.9 | mg/L; |
| L-aspartic acid | 33.3 | mg/L; |
| L-cystine | 42.67 | mg/L; |
| L-cysteine | 68.60 | mg/L; |
| L-glutamic acid | 36.8 | mg/L; |
| L-glutamine | 876 | mg/L; |
| glycine | 26.44 | mg/L; |
| L-histidine | 73.50 | mg/L; |
| L-isoleucine | 89.52 | mg/L; |
| L-leucine | 159.38 | mg/L; |
| L-lysine | 107.21 | mg/L; |
| L-methionine | 87.74 | mg/L; |
| L-phenylalanine | 100.38 | mg/L; |
| L-proline | 96.47 | mg/L; |
| L-serine | 78.46 | mg/L; |
| L-threonine | 136.46 | mg/L; |
| L-tryptophan | 57.81 | mg/L; |
| L-tyrosine | 56.87 | mg/L; |
| L-valine | 96.85 | mg/L; |

nucleotide:

| | | |
|---|---|---|
| hypoxanthine | 10.3 | mg/L; |
| thymidine | 0.24 | mg/L; |
| adenosine | 7 | mg/L; |
| uridine | 7 | mg/L; |
| cytidine | 7 | mg/L; |
| guanosine | 7 | mg/L; |

vitamins:

| | | |
|---|---|---|
| vitamin D | 0.072 | mg/L; |
| folic acid | 5.32 | mg/L; |
| nicotinamide | 3.14 | mg/L; |
| pyridoxine | 3.15 | mg/L; |
| thiamine | 3.23 | mg/L; |
| riboflavin | 0.36 | mg/L; |
| choline chloride | 26.01 | mg/L; |
| D-calcium pantothenate | 5.82 | mg/L; |
| inositol | 25 | mg/L; |

inorganic salts:

| | | |
|---|---|---|
| ferric nitrate | 24.19 | mg/L; |
| ferrous sulfate | 0.417 | mg/L; |
| magnesium sulfate | 48.8 | mg/L; |
| potassium chloride | 311.8 | mg/L; |
| sodium chloride | 6860 | mg/L; |
| disodium hydrogen phosphate | 71 | mg/L; |
| sodium dihydrogen phosphate | 62.5 | mg/L; |
| sodium selenite | 51.88 | mg/L; |

shear force protective agent: 1600 mg/L;
cell clustering resisting agent: 50 mg/L;
pH buffer agent:

| | |
|---|---|
| sodium bicarbonate | 2200 mg/L; |

pH indicator:

| | |
|---|---|
| phenol red | 8 mg/L; |

influenza virus proliferation
accelerant:

| | | |
|---|---|---|
| cholesterol | 3.13 | mg/L; |
| DL-α-tocopherol acetate | 1.39 | mg/L; |
| myristic acid | 0.2284 | mg/L; |
| palmitic acid | 0.256 | mg/L; |
| stearic acid | 0.285 | mg/L; |
| magnesium chloride | 2856 | mg/L; |
| calcium chloride | 174.9 | mg/L; |
| dimethyl sulfoxide | 11 | mg/L; |
| zinc sulfate | 0.8 | mg/L; |
| copper sulfate | 15.97 | mg/L; |
| manganese sulfate | 0.000302 | mg/L; |
| ammonium metavanadate | 0.00234 | mg/L; |

other additives:

| | | |
|---|---|---|
| ammonium ferric citrate | 27 | mg/L; |
| insulin | 6.94 | mg/L; |
| soybean hydrolysate | 2100 | mg/L; |
| ethanolamine | 3.46 | mg/L; |
| glutathione | 1.4 | mg/L. |

3. The serum-free medium for the full suspension culture of the MDCK cells of claim 1, wherein the shear force protective agent is segmented polyether F68.

4. The serum-free medium for the full suspension culture of the MDCK cells of claim 1, wherein the cell clustering resisting agent is dextran sulfate.

5. A preparation method of the serum-free medium for the full suspension culture of the MDCK cells of claim 1, comprising the following steps:
1) preparing a mixed solution: dissolving and mixing raw materials in one of the following methods:
I) mixing and grinding the raw materials into fine powder, and then dissolving the, obtained fine powder in a solvent at 10 to 30° C. to obtain a mixed solution;
II) respectively dissolving the raw materials in the solvent to obtain a raw material solution; and mixing the obtained raw material solutions at a temperature of 10 to 30° C. to obtain a mixed solution; and
2) regulating pH: regulating the pH of the mixed solution to 6.3 to 6.7, and setting a constant volume to obtain the serum-free medium for the full suspension culture of the MDCK cells.

6. The preparation method of the serum-free medium for the full suspension culture of the MDCK cells of claim 5, wherein in step 1), the solvent is pyrogen-free ultra-pure water.

7. The preparation method of the serum-free medium for the full suspension culture of the MDCK cells of claim 5, wherein in step 2), sodium hydroxide is added to regulate the pH value of the obtained mixed solution.

8. A preparation method of the serum-free medium for the full suspension culture of the MDCK cells of claim 2, comprising the following steps:
1) preparing a mixed solution: dissolving and mixing raw materials in one of the following methods:
I) mixing and grinding the raw materials into fine powder, and then dissolving the obtained fine powder in a solvent at 10 to 30° C. to obtain a mixed solution;
II) respectively dissolving the raw materials in the solvent to obtain a raw material solution; and mixing the obtained raw material solutions at a temperature of 10 to 30° C. to obtain a mixed solution; and
2) regulating pH: regulating the pH of the mixed solution to 6.3 to 6.7, and setting a constant volume to obtain the serum-free medium for the full suspension culture of the MDCK cells.

9. The preparation method of the serum-free medium for the full suspension culture, of the MDCK cells of claim 8, wherein in step 1), the solvent is pyrogen-free ultra-pure water.

10. The preparation method of the, serum-free medium for the full suspension culture of the MDCK cells of claim 8, wherein in step 2), sodium hydroxide is added to regulate the pH value of the obtained mixed solution.

11. A preparation method of the serum-free medium for the full suspension culture of the MDCK cells of claim 3, comprising the following steps:
1) preparing a mixed solution: dissolving and mixing raw materials in one of the following methods:
I) mixing and grinding the raw materials into fine powder, and then dissolving the obtained fine powder in a solvent at 10 to 30° C. to obtain a mixed solution;
II) respectively dissolving the raw materials in the solvent to obtain a raw material solution; and mixing the obtained raw material solutions at a temperature of 10 to 30° C. to obtain a mixed solution; and
2) regulating pH: regulating the pH of the mixed solution to 6.3 to 6.7, and setting a constant volume to obtain the serum-free medium for the full suspension culture of the MDCK cells.

12. The preparation method of the serum-free medium for the full suspension culture of the MDCK cells of claim 11, wherein in step 1), the solvent is pyrogen-free ultra-pure water.

13. The preparation method of the serum-free medium for the full suspension culture of the MDCK cells of claim 11, wherein in step 2), sodium hydroxide is added to regulate the pH value of the obtained mixed solution.

14. A preparation method of the serum-free medium for the full suspension culture of the MDCK cells of claim 4, comprising the following steps:
1) preparing a mixed solution: dissolving and mixing raw materials one of the following methods:
I) mixing and grinding the raw materials into fine powder, and then dissolving the obtained fine powder in a solvent at 10 to 30° C. to obtain a mixed solution;
II) respectively dissolving the raw materials in the solvent to obtain a raw material solution; and mixing the obtained raw material solutions at a temperature of 10 to 30° C. to obtain a mixed solution; and
2) regulating pH: regulating the pH of the mixed solution to 6.3 to 6.7, and setting a constant volume to obtain the serum-free medium for the full suspension culture of the MDCK cells.

15. The preparation method of the serum-free medium for the full suspension culture of the MDCK cells of claim 14, wherein in step 1), the solvent is pyrogen-free ultra-pure water.

16. The preparation method of the serum-free medium for the full suspension culture of the MDCK cells of claim 14, wherein in step 2), sodium hydroxide is added to regulate the pH value of the obtained mixed solution.
